(19) United States
(12) Patent Application Publication
(10) Pub. No.: US 2003/0212320 A1
(43) Pub. Date: Nov. 13, 2003

(54) Coordinate registration system for dual modality imaging systems

(76) Inventors: Michael Wilk, Haifa (IL); Binyamin Hajaj, Zoran (IL); Michael Luybansky, Haifa (IL); Hernan Altman, Nesher (IL)

Correspondence Address: DANIEL J SWIRSKY, PO BOX 2345, BEIT SHEMESH, 99544, IL

(21) Appl. No.: 10/260,531

(22) Filed: Oct. 1, 2002

Related U.S. Application Data
(60) Provisional application No. 60/325,580, filed on Oct. 1, 2001.

(51) Int. Cl. A61B 5/05

(52) U.S. Cl. 600/407

(57) ABSTRACT

A method of determining linear and angular displacements of a first coordinate system of a field of view of a first imaging system relative to a second a coordinate system of a field of view of a second imaging system, the method comprising: providing a phantom having a plurality of fiducial regions that can be imaged by both imaging systems; acquiring first and second images of the phantom with the first and second imaging systems wherein spatial coordinates of features in the first and second images reference the first and second coordinate systems respectively; determining positions of a plurality of features of the fiducials in the first image and spatial coordinates of the same features in the second image; and using the coordinates to determine the linear and angular displacements.

### CROSS REFERENCE TO RELATED APPLICATIONS

This application claims priority under 35 U.S.C. § 119(e) to U.S. Provisional Patent Application No. 60/325,580, filed on Oct. 1, 2001, titled “Device and Method of registration for Combined Diagnostic Imaging System,” incorporated herein by reference in its entirety.

### FIELD OF THE INVENTION

The invention relates to imaging systems that employ a plurality of modalities for imaging a patient and in particular to apparatus and methods for registering the fields of view of different sub-systems in the system that image the patient with the different modalities.

### BACKGROUND OF THE INVENTION

In nuclear imaging a radiopharmaceutical that emits radiation is introduced into a patient's body and radiation emitted by the radiopharmaceutical is detected to determine where and in what amounts the radiopharmaceutical concentrates in the body. Depending upon the particular application and pharmaceutical introduced, the detected concentration may be used to generate images of organs and features of the body, or provide functional imaging of the body useable to monitor body processes such as blood flow to an organ or to tag a particular biochemical function. Typical nuclear imaging modalities are single photon emission computerized tomography (SPECT) and positron emission tomography (PET).

While providing generally satisfactory spatial images of concentrations of a radionuclide in a patient's body, nuclear imaging modalities do not in general provide well defined images of the structural anatomy of the patient's body. As a result, a spatial map of the concentration of a radionuclide in a patient provided by a nuclear imaging modality does not in general provide an accurate location of the concentration relative to the morphology of the patient's body.

On the other hand “morphological” imaging modalities, such as CT or MRI, are able to provide satisfactory images of the structural anatomy of the body. To provide improved diagnostic imaging of a patient when nuclear imaging of a patient is desired it is advantageous to image the patient using a nuclear imaging modality in combination with a morphological imaging modality that provides good structural imaging of the patient. An image of the patient provided by the nuclear imaging modality is “fused” with an image of the patient provided by the morphological modality. The fused image provides a spatial map of concentration of a radionuclide located relative to features and organs of the patient's body.

Dual mode (DM) imaging scanners that can acquire images of a patient using both a nuclear imaging modality and a morphological imaging modality and provide a fused image of the patient have been produced. These scanners comprise a sub-system for imaging a patient using a nuclear imaging modality and a sub-system for imaging a patient using a morphological modality. An example of a DM scanner is the GE Discovery LS system, which comprises a CT imaging system and a PET imaging system.

For a DM scanner to provide a properly fused image of a patient from a first nuclear modality image and a second morphological modality image acquired by the scanner, the images should be fused so that points in the images corresponding to same locations in real space are substantially coincident. To an extent that points corresponding to a same real spatial coordinate are coincident, quality of the fused image is better. However, each image is generated with coordinates relative to a coordinate system, hereinafter a “field of view (FOV) coordinate system”, defined by the location and orientation of the field of view of the DM scanner subsystem that is used to acquire the image. The first image is generated with coordinates relative to a first FOV coordinate system defined by the field of view of the nuclear imaging subsystem. The second image is generated with coordinates relative to a second FOV coordinate system defined by the field of view of the morphological imaging subsystem. In general the positions of the different subsystems in a DM scanner are mechanically adjusted and stabilized so that the FOV coordinates systems of their respective fields of view are substantially coincident.

However, accuracy to which FOV coordinate systems of different subsystems in a DM scanner are coincident should be such that a distance between coordinate positions of a same point in real space in different FOV coordinate systems, should be substantially less than the resolution of images provided by the subsystems. In general, as a result for example, of changes in the ambient environment of a DM scanner, vibrations and variable temperature gradients in the scanner, it is often difficult to provide and maintain the required accuracy of coincidence of the FOV coordinate systems of the scanner's imaging subsystems. As a result, quality of fused images provided by a DM scanner is often compromised.

Hereinafter, two FOV coordinate systems that are coincident are said to be registered or “aligned”. Angular and linear displacements of one FOV coordinate system relative to another FOV coordinate system define a degree to which the coordinate systems are mis-aligned or not registered one to the other. The angular and linear displacements are referred to generically as “alignment displacements”.

### SUMMARY OF THE PRESENT INVENTION

An aspect of some embodiments of the present invention relates to improving quality of fused images provided by a DM scanner comprising a first and a second imaging subsystem.

An aspect of some embodiments of the present invention relates to providing methods and apparatus for determining alignment displacements of a first FOV coordinate system of the first imaging subsystem of the DM scanner relative to a second FOV coordinate system of the second imaging subsystem of the DM scanner.

In an embodiment of the present invention a phantom, hereinafter a “calibration phantom”, is scanned by both the first and second subsystems in the DM scanner to provide first and second images respectively of the phantom. The phantom is constructed so that it has regions, hereinafter “fiducial regions” that can be imaged and their geometry and locations in the phantom determined from images acquired by each subsystem. Coordinates of identifiable same features of same fiducial regions in the first and second images are used to determine alignment displacements between the FOV coordinate systems of the first and second imaging subsystems. Such same features may, by way of example, comprise same identifiable points, lines, surfaces and/or volumes of same fiducial regions.

For a SPECT or a PET nuclear imaging subsystems, fiducial regions in a phantom are optionally radioactive regions that emit photons, which are detectable by detectors in the imaging subsystem. Optionally, fiducial regions are regions that are more opaque to photons in the energy bandwidth detected by detectors in the PET or SPECT imaging subsystem than is other material from which the calibration phantom is formed or than is material external to the phantom. To image non-radioactive fiducial regions, at least one radioactive source is mounted to the PET or SPECT imaging subsystem so that when the calibration phantom is located in the field of view of the subsystem, photons from the source pass through the phantom to be incident on detectors in the subsystem. The fiducial regions shadow the source and are detected and their shape delineated by the relatively decreased intensity of photons incident on the detectors along directions that pass through the fiducial regions.

A calibration phantom can also be produced from a material moderately opaque to photons detected by a PET or SPECT imaging subsystem, which is formed with lacunae or voids that function as fiducial regions. The voids, having opacity less than the moderately opaque material are detected by a relatively increased intensity of photons detected by the subsystem along directions that pass through the voids.

Similarly to non-radioactive fiducial regions for PET and SPECT imaging subsystems, fiducial regions comprised in a calibration phantom suitable for CT imaging subsystems, are optionally relatively opaque regions of the phantom. (It is noted that fiducial regions that are relatively opaque regions for a CT imaging subsystem may also be radioactive regions in order to function as fiducial regions for a PET or SPECT imaging subsystem.) As in the case of the PET or SPECT subsystem, voids formed in a material moderately opaque to X-rays from the CT subsystem X-ray source may also function as fiducial regions.

For MRI imaging subsystems regions of a calibration phantom having a relatively high concentrations of atoms characterized by relatively large gyromagnetic ratios may be suitable for fiducial regions. For example, encapsulated volumes of water embedded in porous Styrofoam can function as suitable fiducial regions.

In accordance with some embodiments of the present invention, the position of at least one of the first and second imaging subsystems is adjusted to reduce an alignment displacement between the first and second FOV coordinate systems.

In accordance with some embodiments of the present invention, to generate a fused image from first and second images provided by the first and second imaging subsystems respectively, at least one of the images is transformed responsive to the determined alignment displacements so that coordinates of points in the two images reference substantially a same coordinate system.

There is therefore provided in accordance with an embodiment of the present invention, a method of determining linear and angular displacements of a first coordinate system of a field of view of a first imaging system relative to a second a coordinate system of a field of view of a second imaging system, the method comprising: providing a phantom having a plurality of fiducial regions that can be imaged by both imaging systems; acquiring first and second images of the phantom with the first and second imaging systems wherein spatial coordinates of features in the first and second images reference the first and second coordinate systems respectively; determining positions of a plurality of features of the fiducials in the first image and spatial coordinates of the same features in the second image; and using the coordinates to determine the linear and angular displacements.

Optionally, fiducial regions imaged by the first system are imaged by the second system. Optionally, determining positions of features comprises determining the coordinates to accuracy better than a resolution of an imaging system of the first and second imaging systems having a lowest resolution. Optionally, determining positions of features comprises determining positions for a center of mass for each of the fiducial regions. Optionally, the first and second imaging systems comprise means for adjusting the position of at least one of the systems and comprising controlling the adjustment means responsive to at least one of the determined linear and angular displacements to reduce the at least one displacement.

There is further provided a method of fusing a first image provided by a first imaging system having a first field of view characterized by a first coordinate system and a second image provided by a second imaging system having a second field of view characterized by a second coordinate system, the method comprising: determining at least one of linear and angular displacements that define the position of the first coordinate system relative to the second coordinate system in accordance with a method of the present invention; transforming at least one of the images responsive to the at least one of linear and angular displacements so that coordinates of both images reference a same coordinate system; and fusing the images that reference the same coordinate system.

In some embodiments of the present invention, at least one of the first and second imaging systems is a nuclear imaging system. Optionally, the nuclear imaging system comprises a PET imaging system. Optionally, the nuclear imaging system comprises a SPECT imaging system.

In some embodiments of the present invention, the nuclear imaging system detects photons and the fiducial regions are radioactive regions that emit photons detected by the nuclear imaging system.

In some embodiments of the present invention, the nuclear imaging system detects photons and the fiducial regions are regions that are more opaque to photons in the energy bandwidth detected by the imaging system than is other material from which the phantom is formed and/or than is material external to the phantom and comprising illuminating the phantom with photons that are detected by the nuclear imaging system after passing through the phantom. Optionally, the phantom comprises a relatively low-density material and the fiducial regions are glass spheres embedded in the low-density material.

In some embodiments of the present invention, the phantom comprises a material moderately opaque to photons detected by the nuclear imaging system, which material is formed with voids that function as fiducial regions and comprising illuminating the phantom with photons that are detected by the nuclear imaging system after passing through the phantom.

In some embodiments of the present invention, the at least one of the first and second imaging systems is a CT imaging system. Optionally, the fiducial regions are regions that are more opaque to photons in the energy bandwidth detected by the CT imaging system than is other material from which the phantom is formed and/or than is material external to the phantom. Optionally, the phantom comprises a material formed with voids and wherein the material is moderately opaque to photons detected by the CT imaging system and the voids function as fiducial regions.

In some embodiments of the present invention, the at least one of the first and second imaging systems is an MRI imaging system. Optionally, the fiducial regions are regions of the phantom having relatively high concentrations of atoms characterized by relatively large gyromagnetic ratios. Optionally, the phantom comprises a relatively low-density material and the fiducial regions are encapsulated volumes of water embedded in the low-density material. Optionally, the low-density material is STYROFOAM.

### BRIEF DESCRIPTION OF THE DRAWINGS

The present invention will be understood and appreciated more fully from the following detailed description taken in conjunction with the appended drawings in which

FIGS. 1 and 2 are simplified schematic illustrations of multiple imaging systems arranged for coordinate registration, constructed and operative in accordance with a preferred embodiment of the present invention.

### DETAILED DESCRIPTION OF EXEMPLARY EMBODIMENTS

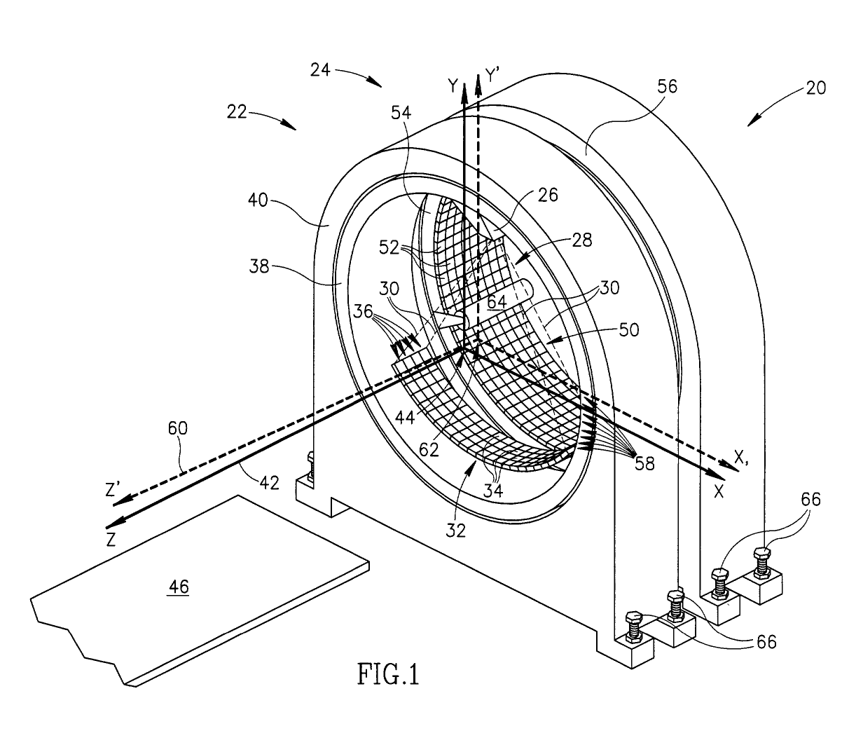

FIG. 1 schematically shows a DM scanner 20 comprising, by way of example, a CT imaging subsystem 22 and a nuclear imaging subsystem 24 which may be a PET, SPECT or PET and SPECT imaging subsystem. Hereinafter it is assumed that imaging subsystem 24 may function as both a PET and SPECT nuclear imager and is referred to as PET-SPECT imaging subsystem 24. Nuclear imaging systems that can function as both PET and SPECT imagers are known in the art. Examples of PET-SPECT imaging systems are found in U.S. Pat. No. 6,255,655 and U.S. Pat. No. 6,303,935. DM scanner 20 fuses images of a patient provided by CT subsystem 22 and PET-SPECT subsystem 24 to provide a fused image of the patient showing concentration of a radiopharmaceutical in the patient's body relative to the structural anatomy of his or her body. Only elements and features of DM scanner 20 and its subsystems 22 and 24 that are germane to the discussion are shown in FIG. 1.

CT imaging subsystem 22 comprises an X-ray source 26 controllable to provide an X-ray fan-beam 28, schematically indicated by dashed lines 30, and an array 32 of X-ray detectors 34 located opposite the X-ray source for sensing intensity of X-rays in fan-beam 28. By way of example CT imagining subsystem 22 is a multislice imaging system and array 32 comprises a plurality of rows 36 of X-ray detectors 34. By way of example in FIG. 1 CT scanner comprises 4 rows 36 of detectors 34. X-ray source 26 and array 32 of X-ray detectors 34 are mounted to a rotor 38 of a gantry 40. Rotor 38 is controllable to be rotated about an axis 42 to rotate X-ray source 26 and detector array 32 about the axis. A volume of space located between detector array 32 and a circle traced out by X-ray source 26 as the X-ray source and detector array are rotated about axis 42 is an imaging field of view (FOV) of CT imaging subsystem 22. Locations in the field of view are defined by (x,y,z) coordinates relative to a FOV coordinate system 44 having it z-axis coincident with axis 42.

A patient to be imaged by CT imaging subsystem 22 is supported on a couch 46. Couch 46 is mounted on a suitable pedestal (not shown) and is controllable to be translated axially along axis 42 so as to move a region of the patient's body to be imaged by CT imaging subsystem 22 through the FOV of the CT subsystem. As the region to be imaged is moved through the FOV of CT subsystem 22, rotor 38 is controlled to rotate X-ray source 26 around axis 42 to illuminate the region with X-rays from a plurality of different angular positions about the patient's body. X-ray “views” of the region acquired at each of the angular positions for which the region is illuminated are processed to provide a CT-image of the region. Coordinates relative to coordinate system 44 define locations of structures and features in the CT-image.

PET-SPECT imaging subsystem 24 comprises an array 50 of gamma detectors 52 mounted in an annulus 54 of a gantry 56. PET-SPECT imaging subsystem 24 is by way of example a full-ring system and array 50 comprises a plurality of full circles 58 of gamma detectors 52. In FIG. 1 PET-SPECT subsystem 24 is shown by way of example comprising 8 circles of gamma detectors 52 and a PET-SPECT subsystem may comprise a number of circles of detectors different from that shown in the figure. In the perspective of FIG. 1 only some of detectors 52 in each circle 58 of detectors 52 is shown.

It is noted that the CT and PET-SPECT imaging subsystems in a DM scanner are not limited to the configurations shown in FIG. 1. For example, PET-SPECT a PET-SPECT imaging subsystem may comprise in place of full circles of detectors, at least one pair of planar detector arrays positioned opposite and facing each other. Similarly a CT subsystem in a DM scanner may be a full ring fourth generation CT-scanner.

A FOV of PET-SPECT imaging subsystem 22 is a volume of space located about an axis 60, which is substantially perpendicular to the planes of detector circles 58 and passes through the center of the circles. Locations in the PET-SPECT FOV are defined by (x′,y′,z′) coordinates relative to a FOV coordinate system 62 having it z′-axis coincident with axis 60.

When a region of a patient lying on couch 46 is to be imaged with PET-SPECT subsystem 24, the couch is moved to position the region of the patient in the FOV of the subsystem. In a PET mode, gamma detectors 52 in array 50 detect pairs of positron-electron annihilation photons emitted from concentrations in the region of a suitable radiopharmaceutical introduced in the patient's body. In a SPECT mode, gamma detectors 52 in array 50 detect single photons emitted by concentrations in the imaged region of a radiopharmaceutical introduced into the patient's body. The numbers of photons detected along different directions along which the photons are emitted from the body are used to provide a spatial PET/SPECT-image of the concentration of the radiopharmaceutical in the region. Coordinates relative to coordinate system 62 define locations of features in the PET/SPECT-image provided by PET-SPECT imaging subsystem 22.

To generate the PET/SPECT image it is advantageous to correct the image for absorption of photons by tissue in the patient's body. Optionally the PET-SPECT subsystem comprises a radiation source 64 that emits photons. The radiation source is mounted to annulus 54 so that it can be rotated about a patient positioned in the FOV of PET-SPECT subsystem 24. As radiation source 64 is rotated about the patient, photons from the radiation source that pass through the patent's body are detected by gamma detectors 52 in array 50. Amounts by which the numbers of photons along different directions through the patient's body are attenuated by the patient's body are used to provide a “transmission image” (similar to a CT image) of the absorption coefficient of the body as a function of position. The absorption coefficient image is used to correct the PET/SPECT image for absorption of photons by tissue in the patient's body. The use of a radiation source to provide data for correcting a PET or SPECT image is known in the art. U.S. Pat. No. 5,210,421, the disclosure of which is incorporated herein by reference, describes using a radiation source for correcting SPECT images.

In order to accurately fuse a CT image of a patient provided by CT subsystem 22 with a PET/SPECT image of the patient provided by PET-SPECT subsystem 24, pixels in each image that image a same real point in space should have coordinates referenced to a substantially same coordinate system. To provide a substantially same coordinate system for images provided by PET-SPECT and CT imaging subsystems in a DM scanner, the DM scanner usually comprises mechanical means for adjusting the positions of the subsystems to improve a degree of registration of the FOV coordinate systems of the subsystems. In DM scanner 20 the mechanical means are represented by jackscrews 66 that can be rotated to adjust the positions of gantries 40 and 56. However, in general as noted above in the background a desired degree of registration between FOV coordinate system 44 and 62 is often not readily obtained and/or maintained. In FIG. 1 FOV coordinate systems 44 and 62 are shown misaligned. The degree of misalignment or lack of registration is exaggerated for clarity of presentation.

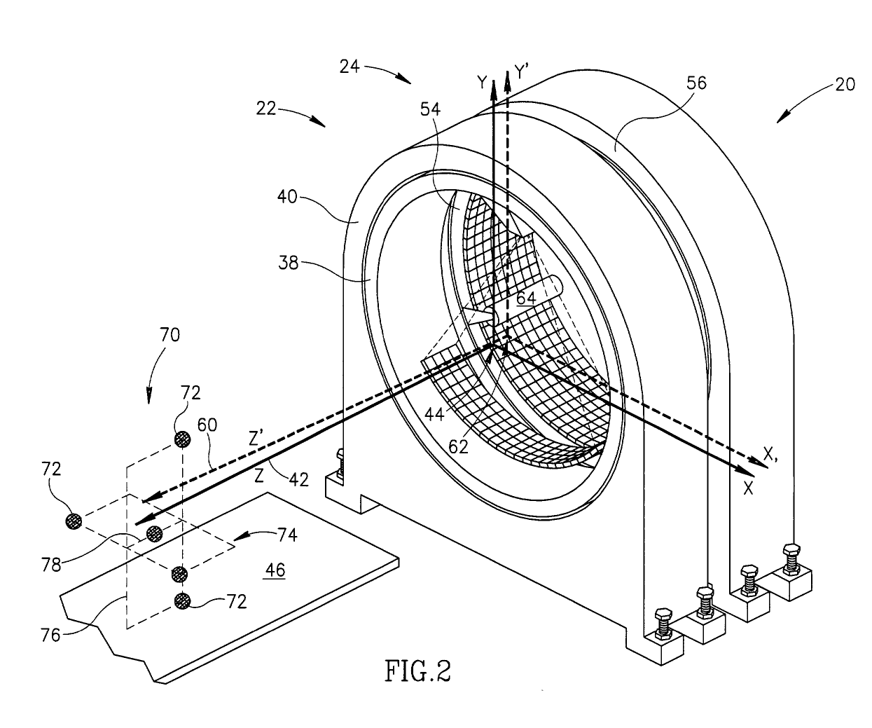

FIG. 2 schematically illustrates a method and apparatus in accordance with an embodiment of the present invention, for determining a degree of alignment displacements that determine a degree of misalignment between FOV coordinate systems 44 and 62 of DM scanner 20.

In FIG. 2 a calibration phantom 70 in accordance with an embodiment of the present invention, is positioned on couch 46. Calibration phantom 70 comprises by way of example five fiducial regions 72 rigidly positioned and maintained relative to each other at different locations by a suitable support material (not shown). Calibration phantom 70 is optionally formed from Styrofoam or a Styrofoam like material and fiducial regions 72 are optionally glass or plastic balls embedded in the Styrofoam. Optionally, three of fiducial regions 72 lie in a first plane indicated by a dashed rectangle 74 and three of the fiducial regions 72 lie in a second plane indicated by a dashed rectangle 76 perpendicular to the first plane. One of the five fiducial regions 72 lies along an intersection 78 of planes 74 and 76. Optionally, fiducial regions 72 in a same plane are not collinear. Optionally, a feature of a fiducial region 72, which is used in accordance with an embodiment of the present invention to determine alignment displacements, is the fiducial region's centers of gravity.

Optionally, fiducial regions 72 have a same diameter. Preferably, the diameter of each fiducial region 72 is such that an image of the fiducial region provided by an imaging subsystem of imaging subsystem 22 and 24 having a lowest resolution contains a plurality of image voxels characteristic of the image. The plurality of image voxels is preferably such that a feature of fiducial region 72 used to determine the alignment displacements (in the present example the fiducial region's center of gravity) can be spatially defined with accuracy sufficient to provide a desired accuracy for values of the alignment displacements. Optionally, the plurality of voxels enables coordinates of the center of gravity to be determined for the “low resolution” imaging subsystem, with accuracy better than a resolution of the subsystem.

Typically, of imaging subsystems 22 and 24, PET-SPECT imaging subsystem 24 is the lower-resolution subsystem. Typically, a PET-SPECT image comprises voxels having a characteristic dimension of about 4 mm, while a CT image has voxels characterized by a dimension of about 1 mm. As a result, in accordance with an embodiment of the present invention, a fiducial region 72 optionally has a diameter such that it contains a plurality of voxels of an image provided by PET-SPECT subsystem 24. Optionally a fiducial region 72 contains at least 8 “PET-SPECT voxels”. Optionally, a fiducial region 72 contains at least 27 PET-SPECT voxels. Optionally, a fiducial region 72 contains at least 64 PET-SPECT voxels.

A maximum distance between two fiducial regions in a same plane 74 or 76 is optionally equal to or greater than about 200 mm. Optionally, the maximum distance is greater than or equal to about 300 mm. Preferably the maximum distance is greater than or equal to about 400 mm. A maximum fiducial distance less than 200 mm may also be used and can be advantageous.

Couch 46 is controlled to move calibration phantom 70 optionally first through the FOV of CT imaging subsystem 22 so that a CT image of the calibration phantom and fiducial regions 72 can be acquired by the subsystem. Couch 46 is optionally controlled to optionally subsequently move calibration phantom 70 through the FOV of PET-SPECT subsystem 24. As calibration phantom 70 is moved through the FOV of PET-SPECT subsystem 24, radioactive source is rotated about the z′-axis. A transmission image is acquired of calibration phantom 70 and fiducial regions 72 by PET-SPECT subsystem 24.

In accordance with an embodiment of the present invention, the CT-image of calibration phantom 70 provided by CT subsystem 22 and the transmission image of the calibration phantom provided by PET-SPECT subsystem 24 are processed to determine coordinates for a center of gravity for each fiducial in each image. If FOV coordinate systems 44 and 62 are coincident, the center of gravity of a same fiducial region 72 has same coordinates in each image. If FOV coordinate systems 44 and 62 are misaligned, the coordinates of the center of gravity of a fiducial region 72 in the CT-image are different from the coordinates of the center of gravity of the fiducial region in the PET-SPECT image. In accordance with an embodiment of the present invention, differences between the coordinates of centers of gravity of same fiducial regions 72 in the CT-image and in the PET-SPECT image are used to determine values for alignment displacements between FOV coordinate systems 44 and 62.

As is well known in the art, six alignment displacements, three linear displacements Δx, Δy, Δz along three orthogonal directions and three rotations Δθ, Δφ, Δξ about orthogonal axes define a position of a first Cartesian coordinate system relative to second coordinate system. The alignment displacements define a linear transform “T(Δx, Δy, Δz, Δθ, Δφ, Δξ)” that transforms coordinates of the first coordinate system into coordinates in the second coordinate system. Coordinates (x,y,z) of a point in FOV coordinate system 44 are therefore related to coordinates (x′,y′,z′) of the point in FOV coordinate system 62 by a transform equation of the form (x,y,z)=T(Δx, Δy, Δz, Δθ, Δφ, Δξ)(x′,y′,z′). Coordinates (x,y,z) are of course transformed to coordinates (x,y,z) by the transform equation (x′,y′,z)=T⁻¹(Δx, Δy, Δz, Δθ, Δφ, Δξ)(x,y,z).

In some embodiments of the present invention, the center of gravity coordinates of fiducial regions 72 in FOV coordinate systems 44 and 62, a chosen one of the preceding transform equations and a least squares procedure are used to determine values for alignment displacements (Δx, Δy, Δz, Δθ, Δφ, Δξ). The determined values are those that minimize least square differences between center of gravity coordinates in one of coordinate systems 44 and 62 and “transformed” center of gravity coordinates in the coordinate system determined by transforming center of gravity coordinates in the other of coordinate systems 44 and 62 using the chosen transform equation.

In some embodiments of the present invention jackscrews 66 are adjusted responsive to determined displacements (Δx, Δy, Δz, Δθ, Δφ, Δξ) to reduce a magnitude of at least one of the values and mechanically improve thereby alignment of FOV coordinate systems 44 and 62. The improved “mechanical alignment” improves quality of images generated by fusing an image provided by CT subsystem 22 with an image provided by PET-SPECT subsystem 24.

In some embodiments of the present invention, the determined displacements (Δx, Δy, Δz, Δθ, Δφ, Δξ) are used to transform the coordinates of one of two images provided by CT subsystem 22 and PET-SPECT subsystem 24 that are to be fused to the coordinates of the other of the images. The transformation of one of the images “mathematically aligns” the two images. Following transformation of the one image, in accordance with an embodiment of the present invention, the two images are fused. The transformation of the one image, in accordance with an embodiment of the present invention, improves quality of the fused image.

The inventors have determined alignment displacements for a CT subsystem and a PET-SPECT subsystem similar respectively to CT subsystem 22 and PET-SPECT subsystem 24 using a calibration phantom similar to calibration phantom 70 and a least squares method. Fiducial regions 72 were glass balls having a diameter of about 16 mm. A maximum spacing between glass balls in a same plane 74 or 76 was between about 300 to about 400 mm. The PET-SPECT subsystem had a resolution of about 4 mm and the CT subsystem had a resolution of about 1 mm. Using mathematical and/or mechanical alignment responsive to the determined alignment displacements, in accordance with an embodiment of the present invention, the inventors found that points in an image provided by the CT subsystem and an image provided by the PET-SPECT subsystem corresponding to a same point in real space could be aligned to within between about ±1.3 mm and about ±2 mm.

Whereas in the above example DM scanner 20 comprises a PET-SPECT subsystem and a CT imaging subsystem, similar methods and apparatus described for aligning images that the subsystems provide and/or their respective FOV coordinate systems are of course applicable to DM scanners comprising a different combination of imaging subsystems. For example, a DM scanner may comprise a PET-SPECT imaging subsystem in combination with an MRI subsystem or a CT subsystem in combination with and an MRI subsystem. Alignment methods and apparatus similar to those described in the above discussion are applicable for these DM scanners. Similar methods and apparatus may also be used to align imaging subsystems in a “multimode” scanner comprising more than two imaging subsystems. It is also noted that whereas the above discussion relates to aligning different mode imaging systems similar alignment methods may of course be used to align same mode imaging systems, such as two CT imaging subsystems in multimode scanner.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A method of determining linear and angular displacements of a first coordinate system of a field of view of a first imaging system relative to a second a coordinate system of a field of view of a second imaging system, the method comprising:
providing a phantom having a plurality of fiducial regions that can be imaged by both imaging systems;
acquiring first and second images of the phantom with the first and second imaging systems wherein spatial coordinates of features in the first and second images reference the first and second coordinate systems respectively;
determining positions of a plurality of features of the fiducials in the first image and spatial coordinates of the same features in the second image; and
using the coordinates to determine the linear and angular displacements.

2. A method according to claim 1 wherein fiducial regions imaged by the first system are imaged by the second system.

3. A method according to claim 1 wherein determining positions of features comprises determining the coordinates to accuracy better than a resolution of an imaging system of the first and second imaging systems having a lowest resolution.

4. A method according to claim 1 wherein determining positions of features comprises determining positions for a center of mass for each of the fiducial regions.

5. A method according to claim 1 wherein the first and second imaging systems comprise means for adjusting the position of at least one of the systems and comprising controlling the adjustment means responsive to at least one of the determined linear and angular displacements to reduce the at least one displacement.

6. A method of fusing a first image provided by a first imaging system having a first field of view characterized by a first coordinate system and a second image provided by a second imaging system having a second field of view characterized by a second coordinate system, the method comprising:
determining at least one of linear and angular displacements that define the position of the first coordinate system relative to the second coordinate system in accordance with claim 1;
transforming at least one of the images responsive to the at least one of linear and angular displacements so that coordinates of both images reference a same coordinate system; and
fusing the images that reference the same coordinate system.

7. A method according to claim 6 wherein at least one of the first and second imaging systems is a nuclear imaging system.

8. A method according to claim 7 wherein the nuclear imaging system comprises a PET imaging system.

9. A method according to claim 7 wherein the nuclear imaging system comprises a SPECT imaging system.

10. A method according to claim 7 wherein the nuclear imaging system detects photons and the fiducial regions are radioactive regions that emit photons detected by the nuclear imaging system.

11. A method according to claim 7 wherein the nuclear imaging system detects photons and the fiducial regions are regions that are more opaque to photons in the energy bandwidth detected by the imaging system than is other material from which the phantom is formed and/or than is material external to the phantom and comprising illuminating the phantom with photons that are detected by the nuclear imaging system after passing through the phantom.

12. A method according to claim 11 wherein the phantom comprises a relatively low-density material and the fiducial regions are glass spheres embedded in the low-density material.

13. A method according to claim 7 wherein the phantom comprises a material moderately opaque to photons detected by the nuclear imaging system, which material is formed with voids that function as fiducial regions and comprising illuminating the phantom with photons that are detected by the nuclear imaging system after passing through the phantom.

14. A method according to claim 6 wherein at least one of the first and second imaging systems is a CT imaging system.

15. A method according to claim 14 wherein the fiducial regions are regions that are more opaque to photons in the energy bandwidth detected by the CT imaging system than is other material from which the phantom is formed and/or than is material external to the phantom.

16. A method according to claim 14 wherein the phantom comprises a material formed with voids and wherein the material is moderately opaque to photons detected by the CT imaging system and the voids function as fiducial regions.

17. A method according to claim 6 wherein at least one of the first and second imaging systems is an MRI imaging system.

18. A method according to claim 17 wherein the fiducial regions are regions of the phantom having relatively high concentrations of atoms characterized by relatively large gyromagnetic ratios

19. A method according to claim 18 wherein the phantom comprises a relatively low-density material and the fiducial regions are encapsulated volumes of water embedded in the low-density material.

20. A method according to claim 19 wherein the low-density material is STYROFOAM.
